(19) United States
(12) Patent Application Publication
(10) Pub. No.: US 2020/0268237 A1
(43) Pub. Date: Aug. 27, 2020

(54) IMAGE OBSERVATION SUPPORT SYSTEM, IMAGE OBSERVATION SUPPORT METHOD AND RECORDING MEDIUM

(71) Applicant: OLYMPUS CORPORATION, Tokyo (JP)

(72) Inventor: Ryota KOBAYASHI, Tokyo (JP)

(73) Assignee: OLYMPUS CORPORATION, Tokyo (JP)

(21) Appl. No.: 16/871,194

(22) Filed: May 11, 2020

Related U.S. Application Data
(63) Continuation of application No. PCT/JP2018/029397, filed on Aug. 6, 2018.

(30) Foreign Application Priority Data
Nov. 13, 2017 (JP) 2017-218615

(51) Int. Cl. A61B 1/04; G06T 7/00; A61B 1/00; G16H 30/40; G16H 50/20; G16H 40/63

(52) U.S. Cl. CPC A61B 1/041; G06T 7/0012; A61B 1/00057; A61B 1/00045; G06T 2207/30028; G16H 50/20; G16H 40/63; G06T 2207/10068; G16H 30/40

(57) ABSTRACT

A position information acquisition unit acquires position information indicating a position of a capsule endoscope occurring at the time of capturing an endoscopic image. A track image generation unit generates track image data representing the movement track of the capsule endoscope inside a subject based on the position information. A display processing unit displays a track image. A shape identification unit identifies an area having a shape that is potentially an obstacle when an insertion part of the endoscope is inserted into the subject.

### CROSS-REFERENCE TO RELATED APPLICATIONS

This application is based upon and claims the benefit of priority from the prior Japanese Patent Application No. 2017-218615, filed on Nov. 13, 2017 and International Application No. PCT/JP2018/029397, filed on Aug. 6, 2018, the entire contents of which are incorporated herein by reference.

### BACKGROUND OF THE INVENTION

### 1. Field of the Invention

The present invention relates to a system for supporting the observation of endoscopic images captured by a capsule endoscope.

### 2. Description of the Related Art

Patent document 1 provides an image display device that allows the user to recognize the movement of a capsule endoscope and the position of the capsule endoscope while the movement and the position are associated with each other. More specifically, Patent document 1 discloses an image display device that acquires position information indicating the position of a capsule endoscope when an internal body image is captured, acquires movement information of the capsule endoscope based on the position information, creates a position model in which an image that schematically represents a passage area of the capsule endoscope is associated with the position of the capsule endoscope at the time of capturing the internal body image, determines a display mode for each area on the position model based on the movement information, and displays the position model in the display mode that has been determined.

[Patent document 1] Japanese Patent Application

Publication No. 2016-63868

In general, a small intestine capsule endoscopic examination is performed after an upper gastrointestinal tract endoscopy or colonoscopy reveals unexplained gastrointestinal bleeding in order to identify the source of the bleeding. When the source of bleeding is found by a capsule endoscopic examination, an endoscopic examination with a treatment tool is performed via an oral route or a transanal route. The doctor determines whether to insert an endoscope via an oral route or a transanal route based on information on the distance between the source of bleeding and the start or end point of the small intestine. More detailed information is preferably provided at the time of the determination.

### SUMMARY OF THE INVENTION

In this background, a purpose of the present invention is to achieve a condition in which a material is provided for the doctor to determine via which route an endoscope is to be inserted.

An endoscopic image observation support system according to one embodiment of the present invention is an observation support system for supporting observation of a plurality of endoscopic images captured inside a subject by a capsule endoscope, including: a position information acquisition unit that acquires position information indicating a position of the capsule endoscope occurring at the time of capturing the images; a track image generation unit that generates track image data indicating a movement track of the capsule endoscope inside the subject based on the position information; and a shape identification unit that identifies an area in the movement track of the capsule endoscope, the area having a predetermined shape.

Optional combinations of the aforementioned constituting elements and implementations of the invention in the form of methods, apparatuses, systems, recording mediums, and computer programs may also be practiced as additional modes of the present invention.

### BRIEF DESCRIPTION OF THE DRAWINGS

Embodiments will now be described, by way of example only, with reference to the accompanying drawings that are meant to be exemplary, not limiting, and wherein like elements are numbered alike in several figures, in which:

FIG. 1 is a diagram for explaining the outline of an image observation support system of a capsule endoscope according to an embodiment;

FIG. 2 is a diagram showing the configuration of a management server and a recorder;

FIG. 3 is a diagram showing an example of an image interpretation screen for an endoscopic image;

FIG. 4 is a diagram showing an example of an overview screen for endoscopic images;

FIG. 5 is a diagram of a digestive tract structure;

FIG. 6 is a diagram showing a display example of a track display area;

FIG. 7 is a diagram showing an example of a selection window;

FIG. 8 is a diagram showing an example of a track image;

FIG. 9 is a diagram showing an example of a track image;

FIG. 10A shows an example of a looped part; and FIG. 10B shows an example of a curved part;

FIGS. 11A and 11B are explanatory diagrams relating to the curved shape of the duodenum; and

FIGS. 12A-12D are diagrams showing examples of a track image.

### DETAILED DESCRIPTION OF THE INVENTION

The invention will now be described by reference to the preferred embodiments. This does not intend to limit the scope of the present invention, but to exemplify the invention.

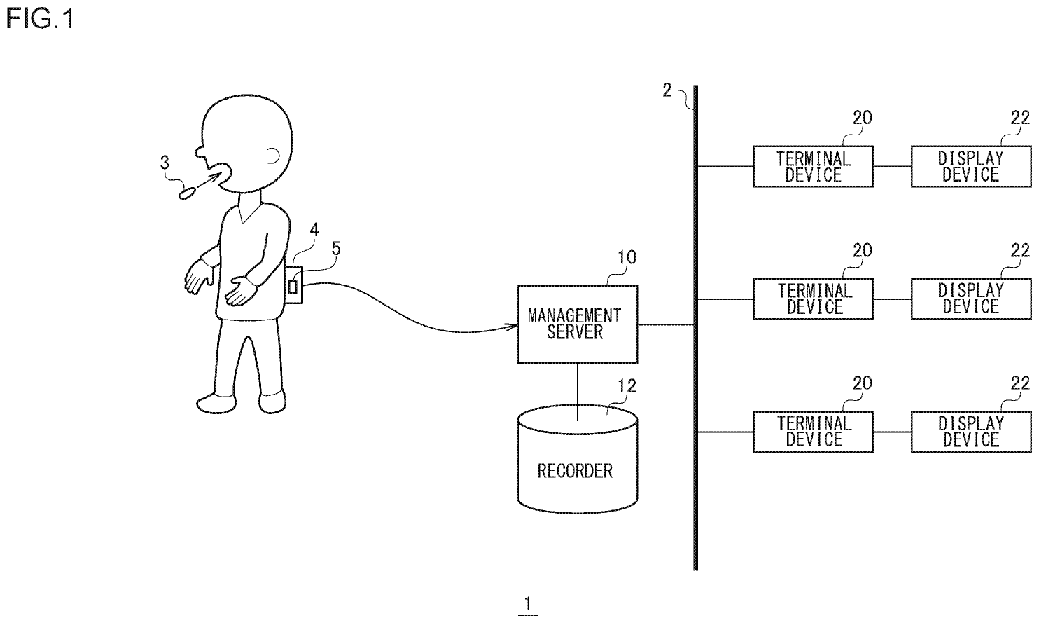

FIG. 1 is a diagram for explaining the outline of an image observation support system of a capsule endoscope according to an embodiment. An endoscopic image observation support system 1 supports observation of a capsule endoscopic image by an image interpreter. In an examination where a usual endoscope is used, a doctor observes an image captured by an endoscope inserted in a patient's body in real time on a display and makes a diagnosis. However, a capsule endoscopic examination is different from a usual endoscopic examination in that the image interpreter collectively observes a large amount of images captured in the past by a capsule endoscope.

In a capsule endoscopic examination, the patient (subject) swallows a capsule endoscope 3 having a built-in ultracompact camera from the mouth, with a plurality of receiver antennas (not shown) being attached to the abdomen and a receiver 4 being attached to the waist by a belt. The capsule endoscope 3 includes: an image capturing unit, which is an ultra-small camera; an illumination unit that illuminates the inside of the subject; a signal processing unit that performs an A/D conversion of an image-capturing signal that is output from the image capturing unit and generates image data to which an image ID and image-capturing time information are added; a memory that temporarily stores image data; a communication module that transmits image data stored in the memory; and a battery that supplies power to each unit. The capsule endoscope 3 captures still images periodically as the capsule endoscope 3 moves through the digestive tract and transmits image data to the receiver 4 through the antennas.

A recording medium 5 is built in the receiver 4, and the receiver 4 adds, to image data received through each receiver antenna, related information including radio wave intensity information at the time of the reception by each receiving antenna and records the image data in the recording medium 5. In the case where the capsule endoscope 3 images the inside of the body every 0.5 seconds, about 60,000 pieces of endoscopic image data are recorded in the recording medium 5 when the image-capturing of the inside of the body is completed in about 8 hours.

The image ID is information for identifying the image and may be information to which a serial number indicating the image-capturing order is assigned. For example, “1” may be assigned to the image ID of an endoscopic image captured first, and “2” may be assigned to the image ID of an endoscopic image captured second. Generating an image ID in this manner allows a serial number included in the image ID to represent the order of capturing images and duplication of image IDs to be avoided. The image ID and the image-capturing time information may be added to the captured image as the related information by the receiver 4 when the receiver 4 receives the captured image. In any case, the image captured by the capsule endoscope 3 is recorded in the recording medium 5 in association with related information such as the image ID, image-capturing time information, and received radio wave intensity.

When the antennas and the receiver 4 are collected from the patient, a data terminal of the receiver 4 becomes connected to a data reader connected to a management server 10, and the data reader reads about 60,000 pieces of endoscopic image data and the related information that are recorded in the recording medium 5 and transmits the endoscopic image data and the information to the management server 10. The data reader may be an external device that becomes connected to the management server 10 by a USB cable or the like. The recording medium 5 may be a memory card detachable from the receiver 4, and the recording medium 5 may be removed from the receiver 4 and installed in the data reader such that the endoscopic image data and the related information are read out. The recording medium 5 may be inserted in a data reading slot provided in the management server 10 such that the endoscopic image data and the related information are read out.

The management server 10 performs a predetermined image process on endoscopic images read from the recording medium 5 and records the images in the recorder 12. The recorder 12 may be comprised of a hard disk drive (HDD) or a flash memory. Since the endoscopic images recorded in the recording medium 5 are uncompressed RAW (raw) images or RAW images on which only lossless compression has been performed, the data size thereof is very large. Therefore, the management server 10 performs a predetermined lossy compression process on the endoscopic images that are RAW images so as to reduce the data size thereof and records the images in the recorder 12. In the embodiment, the management server 10 is in charge of an image process on endoscopic RAW images. However, another device, for example, a terminal device 20 may perform the image process on the endoscopic RAW images and records the images in the recorder 12, and the recorder 12 may be provided in the terminal device 20.

A plurality of terminal devices 20 are connected to the management server 10 via a network 2 such as a local area network (LAN). The terminal devices 20 are personal computers or the like assigned to image interpreters such as doctors and technicians (hereinafter may be simply referred to as “user(s)”) and are connected to display devices 22 to allow an output to be displayed on the respective screens. The terminal devices 20 may be laptop computers integrated with a display device or portable tablets. The terminal devices 20 access the management server 10 to display the endoscopic images recorded in the recorder 12 on the display devices 22.

The management server 10 has a function of executing, when compressing an endoscopic RAW image, an analysis application so as to analyze the endoscopic image. Image analysis by the analysis application is performed on all endoscopic RAW images captured in one capsule endoscopic examination, and the result of the image analysis is added as additional information to the compressed endoscopic images.

One of the purposes of a capsule endoscopic examination is to find the source of bleeding in a digestive tract. Upon acquiring the endoscopic RAW images from the recording medium 5, the management server 10 executes the analysis application so as to perform the image process, thereby identifying an endoscope RAW image in which a bleeding state may have been captured. For example, when the redness in an endoscopic image exceeds a predetermined threshold value, the management server 10 determines that the image is likely to be an image in which a bleeding condition has been captured and adds flag information indicating that the image is a reddish image when compressing the endoscopic RAW image.

The moving speed of the capsule endoscope 3 in a digestive tract varies. Where the moving speed is low, the variation between endoscopic images captured is small. Therefore, it is inefficient and burdensome for an image interpreter to equally observe all the images including a plurality of images with almost no change. In this background, the analysis application compares endoscopic RAW images captured successively in time and performs a process of identifying an image with small change (similar image). Hereinafter, this process will be referred to as “image summarization process”.

In the image summarization process, a reference image is set, and the ratio of a coverage area of the reference image occupying a determination target image subjected to determination as to whether or not the image is similar to the reference image is calculated as a coverage rate. The determination target image is an image captured after the reference image. When the coverage rate is equal to or greater than a threshold value, the analysis application determines the determination target image as a similar image of the reference image. When compressing an endoscopic RAW image serving as the reference image, the management server 10 adds flag information indicating that the image is a reference image, and when compressing an endoscopic RAW image that is a similar image, the management server 10 adds flag information indicating that the image is a similar image.

A playback application executed in the management server 10 or the terminal device 20 has a playback mode for shortening the playback time of endoscopic images with reference to flag information added in the image summarization process. By selecting this playback mode, the image interpreter allows for the shortening of the observation time.

The playback application according to the embodiment has four playback modes.

(First Playback Mode) The first playback mode is a manual playback mode using the operation of a user interface connected to the terminal device 20. In the first playback mode, the user rotates the wheel of a mouse so that the endoscopic images can be frame-by-frame displayed one by one. Therefore, the first playback mode is used for identifying an image in which a pathological change has been captured most distinctly from among a plurality of images in which the pathological change has been captured. When the user rotates the wheel in a direction away from the user, the endoscopic images are continuously played back and displayed in the forward direction (a direction moving from an image with an old image-capturing time toward a new image), and when the user rotates the wheel in a direction toward the user, the endoscopic images are continuously played back and displayed in the backward direction (a direction moving from an image with a new image-capturing time toward an old image).

(Second Playback Mode)

The second playback mode is an automatic playback mode in which the endoscopic images are continuously played back and displayed in the forward direction or the backward direction at a playback speed that has been set. The second playback mode is used for normal endoscopic image observation.

(Third Playback Mode)

The third playback mode is an automatic playback mode in which, while reference images identified through the image summarization process are continuously played back and displayed in the forward direction or the backward direction at a playback speed that has been set, similar images are continuously played back and displayed in the forward direction or the backward direction at a speed higher than the playback speed that has been set. In the third playback mode, by playing back the similar images having a small change from the reference images at a high speed, the shortening of the observation time as compared with the second playback mode can be realized.

(Fourth Playback Mode)

The fourth playback mode is an automatic playback mode in which, while the display of the similar images identified through the image summarization process is omitted, only the reference images are played back and displayed in the forward direction or the backward direction at a playback speed that has been set. In the fourth playback mode, by omitting the display of the similar images, the shortening of the observation time as compared with the third playback mode can be realized. A secondary fourth playback mode, which is a secondary mode of the fourth playback mode may be set. The secondary fourth playback mode is an automatic playback mode in which, while the display of the reference images is omitted, only the similar images are played back and displayed in the forward direction or the backward direction at a playback speed that has been set. The secondary fourth playback mode is used to confirm that there is no omission in observation after the observation in the fourth playback mode.

The first to third playback modes are continuous playback modes for sequentially playing back and displaying endoscopic images that are sequential in time, and the fourth playback mode (the secondary fourth playback mode) is a decimating playback mode where temporally continuous endoscopic images are decimated so as to be played back and displayed. The playback application executes a playback process of the endoscopic images in accordance with a playback mode selected by the user. The playback application may be executed by the management server 10 or may be executed by the terminal device 20.

A user interface such as a keyboard, a mouse, etc., is connected to the terminal device 20. The terminal device 20 has a function of supporting an image interpretation task by the image interpreter in cooperation with the management server 10. The terminal device 20 causes the display device 22 to display an image interpretation screen for endoscopic images, and the user observes the endoscopic images played back and displayed on the image interpretation screen and captures endoscopic images in which pathological changes, etc., have been captured.

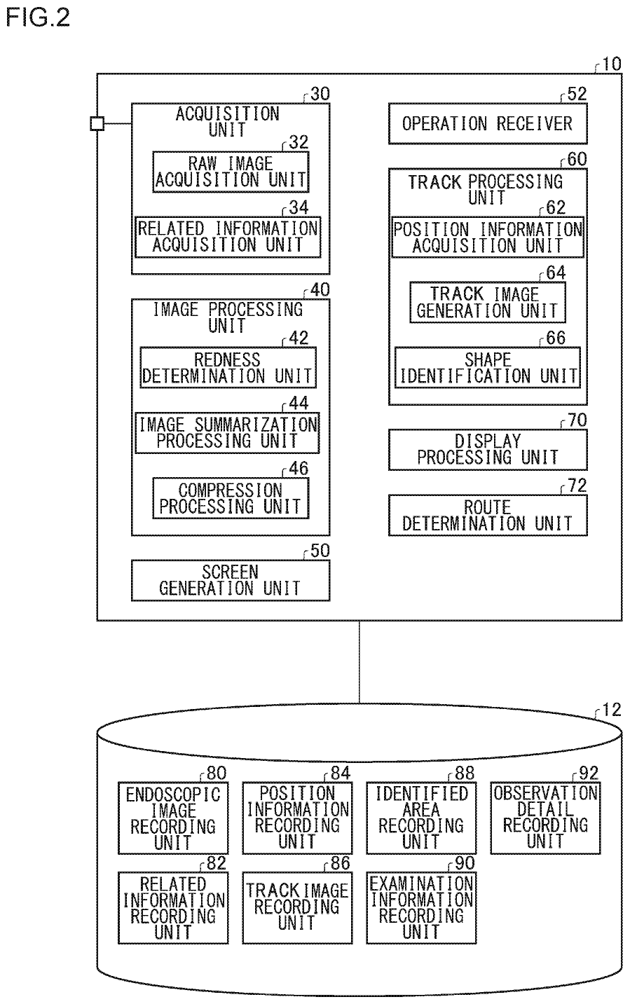

FIG. 2 shows the configuration of the management server 10 and the recorder 12. The management server 10 includes an acquisition unit 30, an image processing unit 40, a screen generation unit 50, an operation receiver 52, a track processing unit 60, a display processing unit 70, and a route determination unit 72. The acquisition unit 30 has a RAW image acquisition unit 32 and a related information acquisition unit 34. The image processing unit 40 has a redness determination unit 42, an image summarization processing unit 44, and a compression processing unit 46. The track processing unit 60 has a position information acquisition unit 62, a track image generation unit 64, and a shape identification unit 66. Each function of the management server 10 may be realized by executing various applications such as an analysis application, a playback application, etc. In the embodiment, the management server 10 executes various applications. Alternatively, the terminal device 20 may execute various applications.

The recorder 12 includes an endoscopic image recording unit 80, a related information recording unit 82, a position information recording unit 84, a track image recording unit 86, an identified area recording unit 88, an examination information recording unit 90, and an observation detail recording unit 92. The endoscopic image recording unit 80 records an endoscopic image on which the image process has been performed by the image processing unit 40. The examination information recording unit 90 records information on an endoscopic examination. The observation detail recording unit 92 records the observation details of the endoscopic image, for example, images captured by the user, information on findings that has been input, and the like.

The configuration of the management server 10 is implemented by hardware such as a processor, a memory, or other LSIs and by software such as a program or the like loaded into the memory. The figure depicts functional blocks implemented by the cooperation of hardware and software. Thus, a person skilled in the art should appreciate that there are many ways of accomplishing these functional blocks in various forms in accordance with the components of hardware only, software only, or the combination of both.

The RAW image acquisition unit 32 acquires about 60,000 endoscope RAW images transmitted from the data reader and temporarily stores the images in the recorder 12. The related information acquisition unit 34 acquires related information transmitted from the data reader and records the related information in the related information recording unit 82. The image processing unit 40 performs the following image process on all the endoscope RAW images. An image ID is associated with endoscopic RAW image data.

<Identification of Reddish Images>

The redness determination unit 42 searches for reddish endoscope RAW images by image analysis and identifies an image with redness that is stronger than a predetermined threshold value. The redness determination unit 42 provides the image ID of the identified reddish image to the compression processing unit 46.

<Image Summarization Process>

The image summarization processing unit 44 performs an image summarization process of grouping all the endoscopic images into reference images and similar images that are similar to the reference image. First, the image summarization processing unit 44 sets the first captured image as a reference image. The image summarization processing unit 44 performs similarity determination as to whether or not the determination target image captured next to the reference image is similar to the reference image. The image summarization processing unit 44 obtains a coverage area including a deformed image obtained by deforming the reference image in the determination target image and calculates, as a coverage rate, the ratio of the coverage area occupying the determination target image.

When the coverage rate is equal to or greater than a threshold value, the image summarization processing unit 44 determines the determination target image as a similar image of the reference image. The image summarization processing unit 44 performs, while using an image captured next to an image determined to be a similar image, similarity determination as to whether or not the image is similar to the reference image. If the moving speed of the capsule endoscope 3 is slow, several tens of images captured after the reference image may be determined as similar images.

On the other hand, when the coverage rate is less than the threshold value, the image summarization processing unit 44 determines the determination target image as a non-similar image. The image summarization processing unit 44 sets an image determined to be a non-similar image as a new reference image and performs similarity determination using, as a determination target image, an image captured next to the image. The image summarization processing unit 44 performs this image summarization process on all the 60,000 or so endoscope RAW images and groups the images into reference images and similar images.

The ratio between the number of the reference images and the number of the similar images is adjusted by the setting of the threshold value. When the threshold value is increased, the number of reference images increases, and when the threshold value is decreased, the number of reference images decreases. In the fourth playback mode, since only reference images are played back and displayed, setting of the threshold value is important for suppressing overlooking of pathological changes and the like. Based on the past results, it has been found that the overlooking of images with pathological changes can be prevented through image interpretation of only the reference images by setting the threshold value such that about 20,000 endoscopic images out of about 60,000 endoscopic images are extracted as the reference images. The image summarization processing unit 44 provides the respective image IDs of the grouped reference images and the respective image IDs of the similar images each to the compression processing unit 46.

<Compression Process on Endoscope RAW Images>

An image analysis process performed by the redness determination unit 42 and the image summarization processing unit 44 is performed at the time of a compression process on endoscopic RAW images performed by the compression processing unit 46. The compression processing unit 46 performs a lossy compression process on an endoscopic RAW image so as to generate an image file to which an image ID and image-capturing time information are added and records the image file in the endoscopic image recording unit 80. For example, the compression processing unit 46 may compress an endoscopic RAW image in an image format such as JPEG.

To the compressed image file, the compression processing unit 46 adds information indicating analysis results provided from the redness determination unit 42 and the image summarization processing unit 44. More specifically, to the compressed image having an image ID provided from the redness determination unit 42, the compression processing unit 46 adds information indicating that the image is a reddish image. This information may be added as flag information. Based on the result of the image summarization process performed by the image summarization processing unit 44, the compression processing unit 46 adds, to a reference image, flag information showing that the image is a reference image and adds, to a similar image, flag information showing that the image is a similar image. Whether an image is a reference image or a similar image is in a front/back relationship. Thus, a flag value 1 may represent a reference image, and a flag value 0 may represent a similar image.

In an embodiment, the redness determination unit 42 and the image summarization processing unit 44 each performs an image process on an endoscopic RAW image before the compression process on the endoscopic RAW image is performed by the compression processing unit 46. In an exemplary variation, the redness determination unit 42 and the image summarization processing unit 44 may each perform image analysis on a compressed image, and information indicating the analysis results may be added to the compressed image. The endoscopic image recording unit 80 records an image file on which the image process has been performed by the image processing unit 40, and the user observes an endoscopic image using the image file recorded in the endoscopic image recording unit 80.

<Locus Process of Capsule Endoscope>

The related information acquisition unit 34 acquires related information transmitted from the data reader and records the related information in the related information recording unit 82. The track processing unit 60 performs a process of identifying the movement track of the capsule endoscope 3 based on the reception intensity information recorded in the related information recording unit 82.

Out of the related information of the image data, the position information acquisition unit 62 acquires position information indicating the position of the capsule endoscope 3 at the time of capturing an endoscopic image, based on the reception intensity information at the time the plurality of receiver antennas have received the image data. This position is a position inside the subject and represents a position when the capsule endoscope 3 captures an image. The method of detecting the image-capturing position of the capsule endoscope 3 is not limited to a method based on the reception intensity of a wireless signal, and a method using a magnetic field or another known method may be used. For example, the position information acquisition unit 62 may derive the moving amount and moving direction inside the subject from the difference between the endoscope image and endoscope images captured before and after the endoscope image, and combine the moving amount and the moving direction inside the subject with the reception intensity information at the plurality of receiver antennas so as to derive position information indicating the position of the capsule endoscope 3 at the time of image capturing. This position information represents position information in a three-dimensional space. The position information acquisition unit 62 records the acquired position information in the position information recording unit 84 in association with the image ID.

The track image generation unit 64 generates track image data representing the movement track of the capsule endoscope 3 inside the subject based on the position information recorded in the position information recording unit 84. The track image generation unit 64 may generate three-dimensional track image data by linking the position information recorded in the position information recording unit 84 in the order of the image ID. At the time of the linking, the track image data may be generated such that connection segments are connected smoothly. The track image generation unit 64 records the generated track image data in the track image recording unit 86.

The shape identification unit 66 identifies an area having a predetermined shape in the movement track of the capsule endoscope 3. As described above, a capsule endoscopic examination is performed for the purpose of searching for the source of unexplained gastrointestinal bleeding, and after the source of bleeding is found, an examination with an endoscope having a treatment tool is performed. In other words, after the source of bleeding is identified, an examination using a normal upper endoscope or lower endoscope having an insertion part and an operation part is scheduled to be performed. As a material for determining whether to insert the endoscope via the oral route or the transanal route, the shape identification unit 66 identifies an area having a shape that is potentially an obstacle when an insertion part of the endoscope is inserted into the subject. A predetermined shape in the movement track may be a looped part that meets a predetermined condition and/or a curved part having a curvature larger than a predetermined value. The shape identification unit 66 records the position of an area indicating the predetermined shape in the movement track in the identified area recording unit 88.

A screen that is displayed on the display device 22 at the time of image interpretation will be described in the following. A doctor B, who is the user, enters the user ID and the password into a terminal device 20 so as to log in. When the user logs in, the management server 10 supplies examination information recorded in the examination information recording unit 90 to the terminal device 20, and the display device 22 displays a list of capsule endoscopic examinations. An examination list screen displays examination information such as the patient ID, the patient name, the examination ID, the date and time of the examination, and the user selects an examination for which an image interpretation report is to be created. When an examination with an examination ID “1111”, a patient name “A”, and an examination ID “0001” is selected from the list of examinations, the screen generation unit 50 generates an image interpretation screen for interpreting an endoscopic image and causes the display device 22 to display the image interpretation screen.

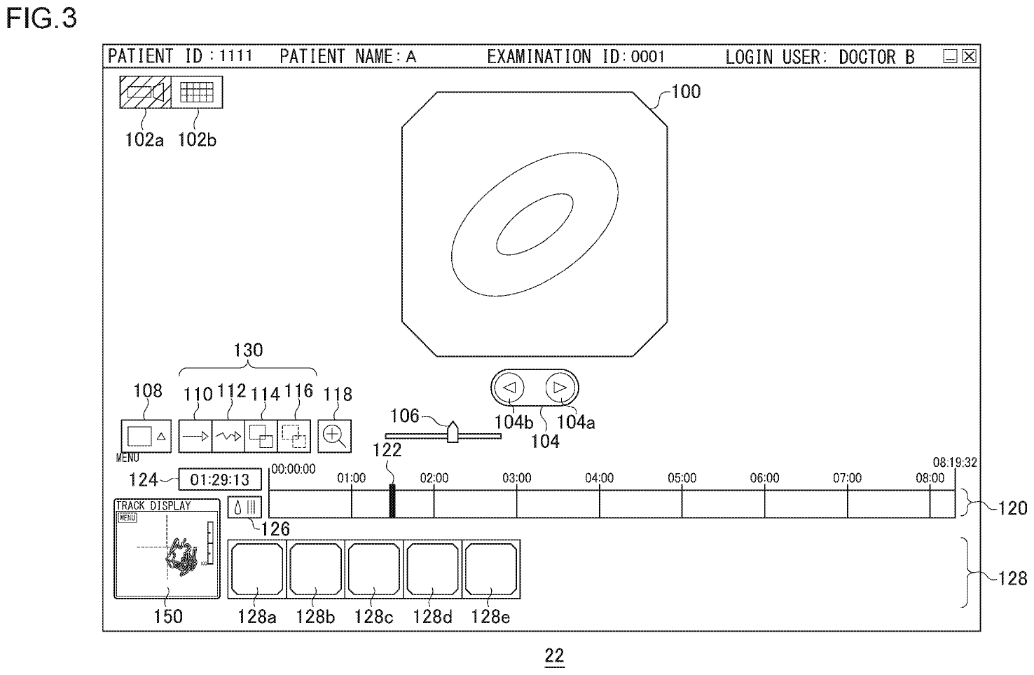

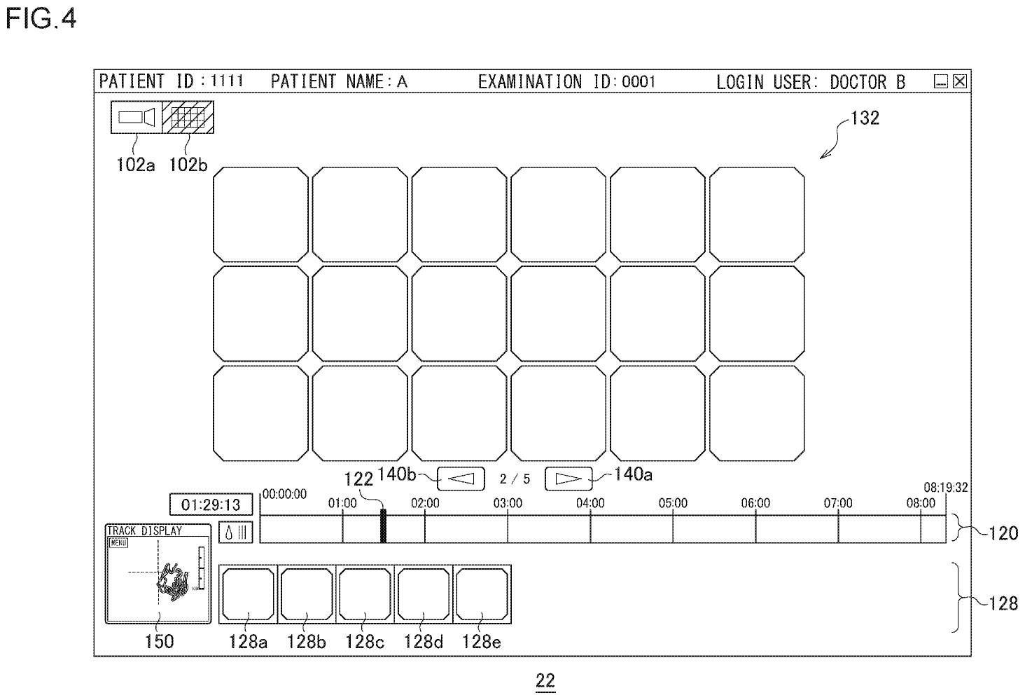

FIG. 3 shows an example of an image interpretation screen for an endoscopic image. A playback area 100 for switching endoscopic images so as to play back and display the endoscopic images is provided at the upper center part of the image interpretation screen. The image interpretation screen is displayed on the display device 22 in a state where a playback mode selection button 102a located at the upper left corner of the screen is being selected. When an overview mode selection button 102b is selected, the screen generation unit 50 generates an overview screen shown in FIG. 4 and displays the overview screen on the display device 22.

A playback number switching button 108 is an operation button for switching the number of images displayed in the playback area 100. Although FIG. 3 shows an example where one-image display is selected, the user can select two-image display or four-image display by operating the playback number switching button 108.

In a playback mode selection area 130, operation buttons for selecting a playback mode are arranged. A second playback mode selection button 110 is an operation button for selecting the second playback mode. A third playback mode selection button 112 is an operation button for selecting the third playback mode. A fourth playback mode selection button 114 is an operation button for selecting the fourth playback mode where only reference images are played back and displayed. A secondary fourth playback mode selection button 116 is an operation button for selecting the secondary fourth playback mode where only similar images are played back and displayed. In the fourth playback mode, since the playback display of similar images is omitted, the user is recommended to observe all the endoscopic images by also performing image interpretation in the secondary fourth playback mode when the user selects the fourth playback mode so as to perform image interpretation.

The user selects one of the second playback mode selection button 110, the third playback mode selection button 112, the fourth playback mode selection button 114, and the secondary fourth playback mode selection button 116 so as to set the playback mode. Under the default state, the second playback mode selection button 110 is selected. A playback button 104a and a reverse playback button 104b are displayed in a playback button display area 104 provided below the playback area 100. When the playback button 104a is selected, endoscopic images are displayed in the forward direction (the direction moving from an image with an old image-capturing time toward a new image) in the playback area 100. When the reverse playback button 104b is selected, the endoscopic images are displayed in the backward direction (the direction moving from an image with a new image-capturing time toward an old image) in the playback area 100. A playback speed adjustment unit 106 includes a slider for adjusting the playback speed (display time for one endoscopic image). The playback speed adjustment unit 106 sets the playback speed, that is, the display frame rate of the endoscopic images, according to the position of the slider.

The display processing unit 70 plays back and displays the endoscopic images in the playback area 100 according to the playback mode selected in a playback mode selection area 130 and the playback speed (display frame rate) set by the playback speed adjustment unit 106. When the playback button 104a or the reverse playback button 104b is selected, the display processing unit 70 starts playing back and displays the images, and a pause button is displayed instead at the place of the playback button 104a or the reverse playback button 104b. When the user operates the pause button during the playing back and displaying of the endoscopic images, the display processing unit 70 pauses the playing back and displaying of the endoscopic images. When the user operates the mouse wheel in this state, the display processing unit 70 displays the endoscopic images frame-by-frame in the first playback mode in accordance with the rotation of the mouse wheel.

When the user places the mouse pointer on an image displayed in the playback area 100 and double-clicks the left button of the mouse, the image is captured and displayed in a captured image display area 128. The captured image displayed in the captured image display area 128 may be selected as an image attached to an image interpretation report later. This example shows a state where five captured images 128a through 128e are selected.

Below the playback area 100, the screen generation unit 50 displays a mark display area 120 with one end indicating the image-capturing start time and the other end indicating the image-capturing end time. In the embodiment, the mark display area 120 is displayed as a time bar with the left end indicating the image-capturing start time and the right end indicating the image-capturing end time, and a slider 122 shows the temporal position of an endoscopic image displayed in the playback area 100. A time position expressed by the slider 122 is also displayed in a time display area 124 as information on relative time from the image-capturing start time. When the user places the mouse pointer on an arbitrary position of the mark display area 120 and clicks the left button of the mouse, an endoscopic image at that time position is displayed in the playback area 100. Even when the user drags the slider 122 and drops the slider 122 at an arbitrary position in the mark display area 120, an endoscopic image at that time position is displayed in the playback area 100.

A red image display button 126 is a button for displaying a red mark for the image-capturing time of a reddish image in the mark display area 120. When the red image display button 126 is operated, the display processing unit 70 displays a red mark for the image-capturing time of a reddish image. By displaying the red mark on the mark display area 120, the user can recognize the presence of an image in which bleeding is highly likely to have been captured.

An enlargement display button 118 is a button for enlarging the playback area 100. When the enlargement display button 118 is operated, the captured image display area 128 is not displayed, and the playback area 100 is enlarged correspondingly.

The user can add a mark for indicating the start position of a site to the mark display area 120. When a new site image is played back while observing endoscopic images played back and displayed in the playback area 100, the user operates a marking button (not shown) to mark the start position of the site on the mark display area 120. By performing this marking process, the start position of a site can be easily known when reviewing endoscopic images. In particular, when a user different from the doctor B observes the endoscope images, the start position of the site can be easily recognized due to the marking process having been performed, and the image observation can be performed smoothly. The marking is generally performed on captured images of the entrance of the stomach, the entrance of the small intestine, and the entrance of the large intestine. Marking information is added to an endoscope image displayed in the playback area 100 when the marking button is operated.

The display processing unit 70 displays a track image in a track display area 150. The track display area 150 is displayed as a window, and the display processing unit 70 can enlarge the track display area 150 through a user operation and move the track display area 150 to an arbitrary position. The display processing unit 70 uses the track image data recorded in the track image recording unit 86 so as to display a three-dimensionally formed track image in the track display area 150. The user can rotate the track image by operating the mouse in the track display area 150. The track image will be described later.

When the overview mode selection button 102b located at the upper left corner of the screen is selected, the screen generation unit 50 generates an overview screen and displays the overview screen on the display device 22. On the overview screen, images extracted from a plurality of reference images identified through the image summarization process are displayed.

FIG. 4 shows an example of the overview screen for endoscopic images. In an image display area 132, images extracted from a plurality of reference images are displayed while being arranged in a lattice pattern. For example, when 20,000 or so reference images are identified from 60,000 or so endoscopic images, the display processing unit 70 displays images extracted at predetermined intervals from the 20,000 or so reference images on the overview screen. The number of images to be extracted may be freely set by the user with an upper limit of 2,000 images. Given that the number of the reference images is N and the number of the images to be included in the overview screen is M, the display processing unit 70 extracts one image for every (N/M) images out of the reference images arranged in a time-series manner. For example, when N equals to 20,000 images and M equals to 2,000 images, the display processing unit 70 extracts one reference image for every 10 images in the order of image-capturing time and arranges the reference image on the overview screen. Extracted images are arranged in a lattice pattern in the image display area 132, and the user can switch images by operating page feeding buttons 140a and 140b.

Since endoscopic images displayed on the overview screen are reference images and are limited to those that are dissimilar to each other, the user can understand the outline of the entire examination efficiently. Further, since the overview screen shows the entire examination in a bird's-eye view, the usability is good when identifying the start position of each site, that is, when performing the marking process. On the overview screen, a function of displaying a plurality of still images temporally preceding and following an image when the user selects the image and performs a predetermined operation is set. Therefore, by selecting the image near the entrance of each site and displaying the images before and after the image, the user can identify the image indicating the start position of each site and perform the marking process efficiently. The user may first perform the marking process on the overview screen shown in FIG. 4 and then observe the image on the interpretation screen shown in FIG. 3.

Referring back to the interpretation screen shown in FIG. 3, the track image displayed in the track display area 150 will be described. As described above, the display processing unit 70 can enlarge and display the track display area 150 through a user operation. For example, the display processing unit 70 may arrange the track display area 150 that is enlarged on the left or right side of the playback area 100.

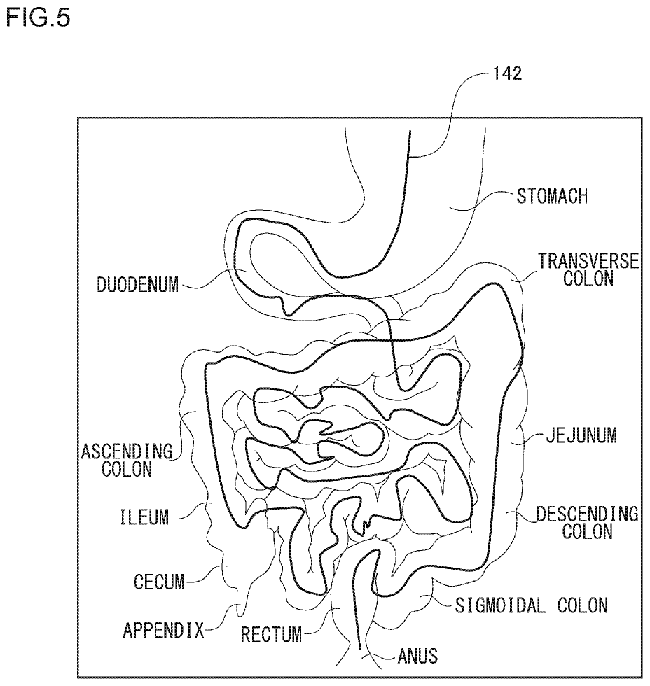

FIG. 5 is a schematic diagram of the digestive tract structure of the stomach, the small intestine, and the large intestine. The capsule endoscope 3 ideally passes through the stomach, the small intestine (duodenum, jejunum, ileum), and large intestine and is discharged from the anus in a track shown by a track curve 142. On the other hand, since the small intestine is an organ that is not fixed in the body, the shape of the small intestine often differs from the ideal shape, and depending on the subject, the intestinal tract may form a loop due to adhesions, making the shape thereof to differ greatly from the ideal shape. Further, since the capsule endoscope 3 is passively moved by the peristaltic motion of the digestive tract, the capsule endoscope 3 may move back and forth at the same place or may stay. Therefore, the actual movement track of the capsule endoscope 3 often does not show an ideal curve such as the track curve 142.

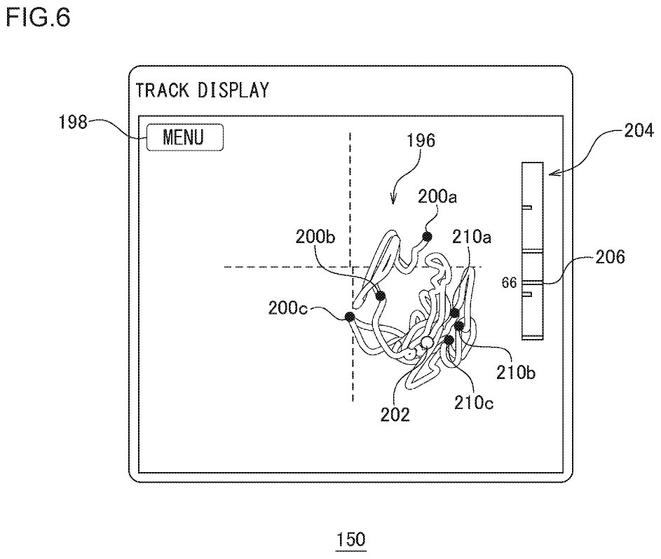

FIG. 6 shows an example of a track display area 150 enlarged next to the playback area 100. The display processing unit 70 reads out the track image data recorded in the track image recording unit 86 and displays the track image 196 three-dimensionally in the track display area 150. Displaying the track image 196 three-dimensionally means that the user can rotate the track image 196 by dragging the track image 196 using the mouse.

In the track image 196, a current position mark 202 indicates a position on the track of the endoscope image played back and displayed in the playback area 100. The user can move the current position mark 202 by operating the mouse. When the user drags the current position mark 202 with the mouse and drops the current position mark 202 at another position on the track, an endoscope image corresponding to the position at which the current position mark 202 has been dropped is displayed in the playback area 100.

An intraintestinal position gauge 204 is a scale indicating a relative distance (position) in the entire small intestine. The upper end (scale 0) indicates the small intestine start position, and the lower end (scale 100) indicates the small intestine end position. A current position indicator 206 indicates the relative distance in the entire small intestine. In the illustrated example, the current position indicator 206 of “66” indicates that the endoscope image displayed in the playback area 100 is an image captured at a position that is two thirds away in a small intestinal section from the small intestine start position. When the user moves the current position mark 202 to another position on the track as described above, the current position indicator 206 on the intraintestinal position gauge 204 also moves to the corresponding position. If the current position mark 202 is in a stomach section, the current position indicator 206 indicates “0”, and if the current position mark 202 is in a large intestine section, the current position indicator 206 indicates “100”.

The display processing unit 70 adds a site mark 200 indicating the position where a site is marked to the track image 196. In the example shown in FIG. 6, a stomach start mark 200a indicates the stomach start position, a small intestine start mark 200b indicates the small intestine start position, and a large intestine start mark 200c indicates the large intestine start position. The display processing unit 70 displays the stomach section between the stomach start mark 200a and the small intestine start mark 200b, the small intestine section between the small intestine start mark 200b and the large intestine start mark 200c, and the large intestine section at and after the large intestine start mark 200c in different colors. Thereby, the user can confirm the shape of each section at a glance.

If the doctor has already entered finding information in association with the endoscope image in the form of a report, the display processing unit 70 may refer to the finding information recorded in the observation detail recording unit 92 so as to add a finding mark 210 indicating the endoscope image linked to each finding to the track image 196. In this example, a first finding mark 210a, a second finding mark 210b, and a third finding mark 210c indicate the positions on the track of the respective endoscope images respectively linked to three findings entered by the doctor. When the user aligns the current position mark 202 with a finding mark 210 by operating the mouse, the display processing unit 70 displays an endoscope image (an image linked to the finding) captured at the position in the playback area 100.

In the embodiment, various display settings of a track image can be made, thereby effectively supporting the user to observe endoscope images. A menu button 198 is a button for displaying a setting item selected by the user and is provided in the track display area 150. The menu button 198 may be provided in an area other than the track display area 150.

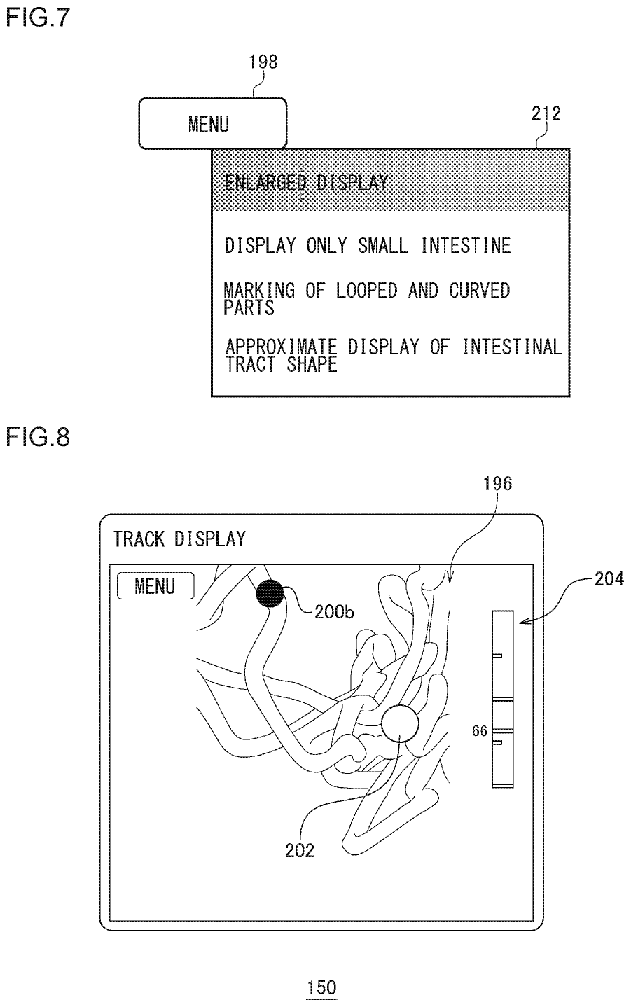

FIG. 7 shows an example of a selection window 212 displayed when the menu button 198 is operated. In the selection window, items: “enlarged display”; “display only small intestine”; “marking of looped and curved parts”; and “approximate display of intestinal tract shape”, are prepared. When the user positions the cursor on one item and left-clicks, the display processing unit 70 performs display processing of the corresponding item.

FIG. 8 shows a track image displayed when “enlarged display” is selected in the menu window. When the operation receiver 52 receives a selection operation of “enlarged display”, the display processing unit 70 displays the track image 196 in an enlarged manner. This allows the user to closely observe the shape of the intestinal tract. When the operation receiver 52 receives an operation for specifying an enlargement position from the user, the display processing unit 70 determines a range for enlarged display according to the operation of the user.

When the operation receiver 52 receives the selection operation of “display only small intestine”, the display processing unit 70 extracts only the small intestinal section between the small intestine start mark 200b and the large intestine start mark 200c from the track image 196, and displays the small intestinal section in the track display area 150.

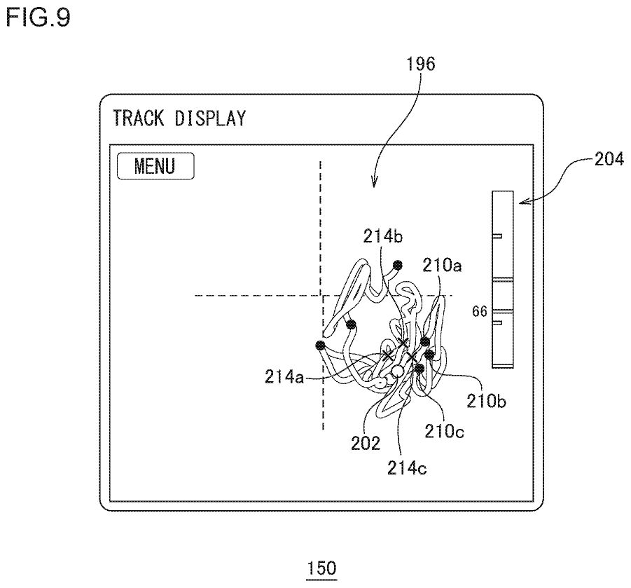

FIG. 9 shows a track image displayed when “marking of looped and curved parts” is selected in the menu window. As described above, the shape identification unit 66 identifies an area having a predetermined shape in the movement track of the capsule endoscope 3, and records the position of the identified area in the identified area recording unit 88. The region having a specific shape is an area having a shape that is potentially an obstacle to insertion of a normal endoscope that is not a capsule endoscope, that is, an endoscope that captures an internal body image through the insertion of an insertion part into the body and the operation of the operation part by a doctor. For example, the area is an area where the intestinal tract is connected due to adhesion or an area where the shape of the intestinal tract is not normal due to a congenital abnormality. In the embodiment, the shape identification unit 66 analyzes the track image data so as to detect a looped part that meets a predetermined condition and/or a curved part having a curvature larger than a predetermined value.

<Looped Part>

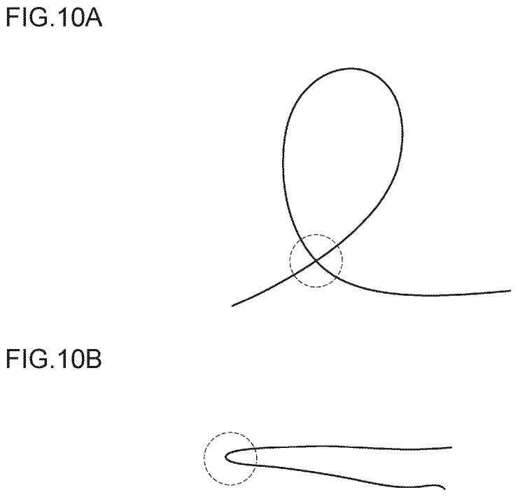

FIG. 10A shows an example of a looped part making a full circle with a small diameter in the movement track. When the track image is viewed from a predetermined direction, the intestinal tract crosses itself. The shape identification unit 66 analyzes the track image data, detects the presence of a looped part that meets a predetermined condition, and identifies the position of the looped part. At the crossing point, it is assumed that a distance between two positions overlapping in a predetermined direction is D and that a difference between arrival times of the capsule endoscope 3 at the two positions is T.

D<d (1)

t1<T<t2 (2)

When both of the above conditions are satisfied, the shape identification unit 66 detects an area including the crossing point as a looped part.

The condition (1) requires the distance between the two positions at a point where the intestinal tract crosses itself to be short. Even when the intestinal tract crosses itself, as long as the distance between the two positions at the crossing point is long, such an area does not become an obstacle to the insertion of the endoscope and thus does not need to be detected as a looped part. For example, d may be about 5 cm.

In the condition (2), t1<T requires the time T spent for returning to the crossing point to be longer than the predetermined time t1 and is a condition required for excluding a case where the capsule endoscope 3 moves back and forth at a similar place due to the peristaltic motion. On the other hand, T<t2 requires the time T spent for returning to the crossing point to be shorter than the predetermined time t2 and is a condition required for excluding a case where the capsule endoscope 3 does not move due to staying. For example, t1 may be about 10 seconds, and t2 may be about 60 seconds.

The shape identification unit 66 detects an area satisfying the conditions (1) and (2) as a looped part and records position information indicating a representative position of the area in the identified area recording unit 88.

<Curved Part>

FIG. 10B shows an example of a curved part in the movement track of the capsule endoscope 3. The shape identification unit 66 analyzes the track image data, detects an intestinal tract shape forming an acute angle equal to or smaller than a predetermined value as a curved part, and identifies the position. The shape identification unit 66 records the position information of the identified curved part in the identified area recording unit 88.

Although irrelevant to insertion difficulty, the small intestine has a characteristic curved shape, which is the duodenal C-shape. Since there are many pathological changes that occur near the Vater's papilla, some doctors desire to carefully make observations. Therefore, the shape identification unit 66 may identify the C-shape of the duodenum in advance, and the display processing unit 70 may notify the user of the position of the C-shape.

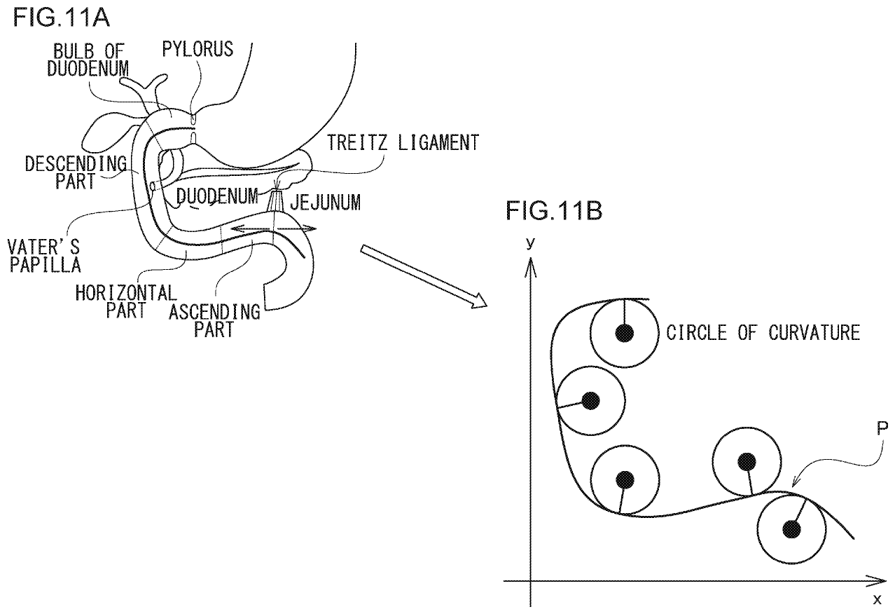

FIG. 11A is a schematic diagram of the curved shape of the duodenum. In the figure, the intestinal tract from the stomach, to the duodenum, and to the jejunum is shown. FIG. 11B is a diagram for explaining a method for identifying the curved shape of the duodenum. In FIG. 11B, for an arithmetic process, the track of the capsule endoscope 3 moving the intestinal tract from the stomach, to the duodenum, and to the jejunum is developed on xy coordinates.

For the identification of the C-shape of the duodenum, the features of the Treitz ligament, which is the only ligament that suspends the small intestine, are used. The small intestine is divided into the duodenum and the jejunum before and after the Treitz ligament. The jejunum is arranged such that the jejunum goes downward after the Treitz ligament. Therefore, as shown in FIG. 11B, when the circle of curvature is set for the movement track, the y coordinate of the center of the circle of curvature is larger than the Y coordinate of the track position (the circle of curvature is set above the track) before a point P of the Treitz ligament, and the y coordinate of the center of the circle of curvature is smaller than the Y coordinate of the track position (the circle of curvature is set below the track) after the point P. By using this property, the shape identification unit 66 may identify the position of the Treitz ligament in the C-shape of the duodenum and record the position in the identified area recording unit 88.

Referring back to FIG. 9, the display processing unit 70 adds an insertion obstruction mark 214 to the track image 196 with reference to the position information recorded in the identified area recording unit 88. In this example, a first insertion obstruction mark 214a, a second insertion obstruction mark 214b, and a third insertion obstruction mark 214c are added to the track image 196 as information indicating a position that may obstruct insertion of the endoscope. By looking at an insertion obstruction mark 214, the user can determine on which side, whether the oral side or the anal side, the endoscope can be easily inserted into the subject up to the position of a pathological change. In the example shown in FIG. 9, three finding marks 210 are added to the track image 196. For example, when all the three insertion obstruction marks 214 are located upstream (oral side) of the image position to which each finding mark 210 is added, it is easy to determine that it is better to insert the endoscope from the anal side. As described above, the display processing unit 70 displays the finding marks 210 and the insertion obstruction marks 214 together in the track image 196, thereby allowing the user to determine the relative location of a part that causes the insertion to be difficult with respect to the position of a pathological change.

Based on the position of the area identified by the shape identification unit 66, the route determination unit 72 may automatically determine on which side, whether the oral side or the anal side, the endoscope can be easily inserted into the subject up to the position of a pathological change. For example, based on the relative positional relationship between the position of a pathological change and an insertion obstruction position and on the insertion difficulty due to each insertion obstacle, the route determination unit 72 derives a difficulty value when the endoscope is inserted from the oral side and a difficulty value when the endoscope is inserted from the anal side and determines an insertion route by comparing the difficulty values. At this time, information on the distance from the small intestine start position or the small intestine end position may be added. For example, when there are a plurality of positions of pathological changes, the insertion difficulty may be derived for each of the positions, and the highest difficulty value when the endoscope is inserted from the oral side and the highest difficulty value when the endoscope is inserted from the anal side may be compared so as to select the one showing the lower difficulty value as the insertion route.

The display processing unit 70 may indicate the position of the curved shape of the duodenum described above in the track image 196. In this case, since the curved shape of the duodenum does not represent the degree of ease of insertion of the endoscope, a mark different from the insertion obstruction marks 214 is preferably added to the track image 196.

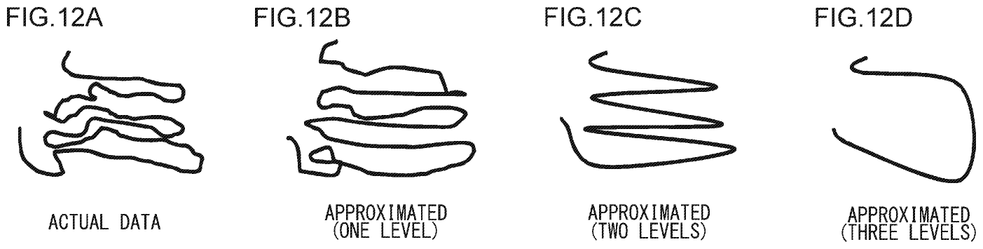

FIG. 12 show examples of a track image displayed when “approximate display of intestinal tract shape” is selected in the menu window. FIG. 12A is a track image displaying the track image data recorded in the track image recording unit 86. FIG. 12B is a track image approximated to smooth the track image data by one level. FIG. 12C shows a track image approximated to smooth the track image data by two levels. FIG. 12D shows a track image approximated to smooth the track image data by three levels. The level of an approximate image corresponds to the approximation ratio, and the user can set the approximation ratio and display a desired approximate image. The display processing unit 70 creates an approximate image of the track image data using the least square method or the like according to the approximation ratio that is set, and displays the approximate image in the track display area 150.

The user can learn a rough intestinal tract shape based on the approximate image. In particular, a rougher intestinal tract shape can be learned as the approximation ratio is lowered. For example, in a case of a congenital intestinal abnormality, the shape of the intestinal tract may be completely different from the standard one (for example, a left and right reversed case). It is meaningful to obtain such information in advance when inserting an endoscope.

Described above is an explanation based on the embodiments of the present invention. These embodiments are intended to be illustrative only, and it will be obvious to those skilled in the art that various modifications to constituting elements and processes could be developed and that such modifications are also within the scope of the present invention.

What is claimed is:

1. An image observation support system for supporting observation of a plurality of images captured inside a subject by a capsule endoscope, comprising:
a processor comprising hardware, wherein the processor is configured to:
acquire position information indicating a position of the capsule endoscope occurring at the time of capturing the images;
generate track image data indicating a movement track of the capsule endoscope based on the position information; and
identify an area in the movement track, the area having a shape that is an obstacle when the endoscope is inserted into the subject.

2. The image observation support system according to claim 1, wherein
the processor identifies a position of a looped part that meets a predetermined condition and/or a curved part having a curvature of a predetermined value or greater.

3. The image observation support system according to claim 1, wherein
the processor displays a track image, and a predetermined mark at the position of the area in the track image.

4. The image observation support system according to claim 3, wherein
the processor displays an approximate image of the track image.

5. The image observation support system according to claim 1, wherein
the processor determines on which side, whether the oral side or the anal side, the endoscope can be easily inserted into the subject up to the position of a pathological change, based on the position of the area.

6. An image observation support method for supporting observation of a plurality of images captured inside a subject by a capsule endoscope, comprising:
acquiring position information indicating a position of the capsule endoscope occurring at the time of capturing the images;
generating track image data indicating a movement track of the capsule endoscope based on the position information; and
identifying an area in the movement track, the area having a shape that is an obstacle when the endoscope is inserted into the subject.

7. The image observation support method according to claim 6, wherein
the area that is identified is a looped part that meets a predetermined condition and/or a curved part having a curvature of a predetermined value or greater.

8. The image observation support method according to claim 6, comprising:
displaying a predetermined mark at the position of the area in a track image when displaying the track image.

9. The image observation support method according to claim 8, comprising:
displaying an approximate image of the track image.

10. The image observation support method according to claim 6, comprising:
determining on which side, whether the oral side or the anal side, the endoscope can be easily inserted into the subject up to the position of a pathological change, based on the position of the area.

11. A non-transitory computer readable recording medium having recorded thereon a program for supporting observation of a plurality of images captured inside a subject by a capsule endoscope, wherein the program includes:
a module that acquires position information indicating a position of the capsule endoscope occurring at the time of capturing the images;
a module that generates track image data indicating a movement track of the capsule endoscope based on the position information; and
a module that identifies an area in the movement track, the area having a shape that is an obstacle when the endoscope is inserted into the subject.

12. The recording medium according to claim 11, wherein
the area that is identified is a looped part that meets a predetermined condition and/or a curved part having a curvature of a predetermined value or greater.

13. The recording medium according to claim 11, wherein
the program displays a predetermined mark at the position of the area in a track image when displaying the track image.

14. The recording medium according to claim 13, wherein
the program displays an approximate image of the track image.

15. The recording medium according to claim 11, wherein
the program determines on which side, whether the oral side or the anal side, the endoscope can be easily inserted into the subject up to the position of a pathological change, based on the position of the area.
